(19) United States
(12) Patent Application Publication
(10) Pub. No.: US 2011/0280931 A1
(43) Pub. Date: Nov. 17, 2011

(54) MATRIX METALLOPROTEINASE INHIBITOR

(76) Inventor: Curt Harold Grob, Mount Pleasant, SC (US)

(21) Appl. No.: 12/778,161

(22) Filed: May 12, 2010

(51) Int. Cl. A61K 31/215; C07C 229/46; A61K 9/127; A61P 35/00

(52) U.S. Cl. 424/450; 514/529; 560/125

(57) ABSTRACT

Compounds of FIG. 3:

###FIG. 3###

Whereby R and R1 preferentially correspond to ring structure consisting of FIG. :

###FIG. 4###

Whereby R or R1, S or S1 is a Carbon, Nitrogen or Sulfur atom Z or Z1 and B or B1 is independently H or an alkyl, amino acid residue or amide thereof. Alternatively, Z or Z1 and B or B1 may constitute an fused, unsubstituted, cyclic ring structure, a cyclic amine or heterocyclic amine, or heavy metal, halogen or oxygen species. Compounds and derivatives are useful in treating conditions whereby matrix metalloproteinase activity is not desired, and inhibition is therapeutically beneficial.

### CROSS REFERENCE TO RELATED APPLICATION

This application is a continuation-in-part of U.S. provisional patent No. 60885883 filed Jan. 20, 2007 now abandoned.

US Patent Documents

| | | | |
|---|---|---|---|
| 6,579,982 | July 2003 | Blagg | 544/300 |
| 5,872,152 | February 1999 | Brown et. al | 514/575 |
| 6,818,622 | November 2004 | Bird et al. | 514/19 |
| 6,352,976 | March 2002 | Warshawsky et. al | 514/18 |
| 6,930,097 | August 2005 | Hsu et al. | 514/19 |
| 6,063,786 | May 2000 | Baxter et al. | 514/272 |
| 6,683,060 | January 2004 | Hsu et al. | 514/49 |
| 5,847,153 | December 1998 | Warpehoski et al. | 548/319.5 |
| 4,558,034 | December 1985 | Galardy et al. | 514/7 |
| 4,599,361 | July 1986 | Dickens et al. | 514/575 |
| 4,681,894 | July 1987 | Murray et al. | 514/507 |
| 4,743,587 | May 1988 | Dickns et al. | 514/575 |
| 4,918,105 | April 1990 | Cartwright et al. | 514/575 |
| 5,616,605 | April 1997 | Gray et al. | 514/415 |
| 5,892,112 | April 1999 | Levy et al. | 564/133 |
| 5,932,595 | August 1999 | Bender et al. | 514/317 |
| 6,013,649 | January 2000 | Freskos et al. | 514/237.8 |
| 6,118,001 | September 2000 | Owen et al. | 546/229 |
| 6,380,258 | April 2002 | Bedell et al. | 514/575 |
| 6,465,468 | October 2002 | Baxter et al. | 514/309 |
| 5,183,900 | February 1993 | Galardy et al. | 548/495 |
| 5,189,178 | February 1993 | Galardy et al. | 548/495 |
| 4,595,700 | June 1986 | Donald et al. | 514/616 |
| 6,541,489 | April 2003 | Barta et al. | 514/300 |

US Cl. 514/19, 514/2, 514/6, 530/868, 260/998.2, 260/998.21, 514/575, 514/300
Field of Search 514/19, 2, 6; 260/998.2, 260/998.21, 530/868, 514/575

### OTHER PUBLICATIONS CITED

Rodriguez J A, Orbe J, Paramo J A. Metalloproteinases, vascular remodeling and atherothrombotic syndromes. Rev Esp Cardiol. 2007 September; 60 (9):959-67.
Wallach J, Hornebeck W: Matrix metalloproteinases and cancer. Novel perspectives in the role and control of matrix metalloproteinases in tumor progression. Biochimie 87: 241, 2005
Fingleton B: Matrix metalloproteinases: Roles in cancer and metastasis. Front Biosci 11: 479-491, 2006
Handsley M M, Edwards D R: Metalloproteinases and their inhibitors in tumor angiogenesis. Int J Cancer 115: 849-860, 2005
Roomi M W et al. “A Novel in Vitro Bioassay for Screening Matrix Metalloproteinase Activity in Human Cancer Cell Lines (2003)” http://www.drrathresearch.org/lab_research/study_c_a_novel_invitro.html Downloaded on Oct. 7, 2007.
Green M J, Gough A K S, Devlin J, et al. Serum MMP-3 and MMP-1 and progression of joint damage in early rheumatoid arthritis. Rheumatology 2003; 42:83-88.
Stamenkovic I. Extracellular matrix remodelling: the role of matrix metalloproteinases. J Pathol. 2003 July; 200 (4):448-64.
Konttinen Y T, Ainola M, Valleala H, et al. Analysis of 16 different matrix metalloproteinases (MMP-1 to MMP-20) in the synovial membrane: different profile in trauma and rheumatoid arthritis. Ann Rheum Dis 1999; 58:691-697.
Ramnath N, Creaven P J: Matrix metalloproteinase inhibitors. Curr Oncol Rep 6: 96-102, 2004
Skiles J W, Gonnella N C, Jeng A Y. The design, structure, and clinical update of small molecular weight matrix metalloproteinase inhibitors. Curr Med Chem 2004 November; 11 (22):2911-77.
Beckett, et al., “Recent Advances in Matrix Metalloproteinase Inhibitor Research” Drug Discovery Today, 1996, 16-26, vol 1, No 1.
Whittaker M, Floyd C D, Brown P, Gearing A J. Design and therapeutic application of matrix metalloproteinase inhibitors.
Chem Rev. 1999 Sep. 8; 99 (9):2735-76.
Michaelides, M R et al. Recent advances in matrix metalloproteinase inhibitors research. Curr Pharm Design 5, 787-819, 1999.
Hoekstra R, Eskens FALM, Verweij J. Matrix Metalloproteinase Inhibitors: Current Developments and Future Perspectives. The Oncologist 2001; 6:415-427.
Kaneda N, Nagata H, Furuta T, Yokokura T. Metabolism and pharmacokinetics of the camptothecin analogue CPT-11 in the mouse. Cancer Res 1990; 50: 1715-1720.
Ohe. Y, Sasaki Y, Shinkai T et al. Phase I study and pharmacokinetics of CPT-11 with 5-day continuous infusion. J Natl Cancer Inst 1992; 84: 972-974.
Morrissey Lab Protocol for Preparing Phospholipid Vesicles (SUV) by Sonication, James H. Morrissey 2001, University of Illinois at Urbana-Champaign
Betageri G V, Jenkins S A and Parsons D L. Liposome Drug Delivery Systems. Technomic Publishing Company Inc., USA, Lancaster, Pa., 1993.

This invention was not developed with federally sponsored research or development.

### TECHNICAL FIELD

This invention is directed to pharmaceuticals that are useful in diseases characterized by undesired matrix metalloproteinase activity. More specifically, the invention concerns compounds such as N-cyclohexanehydroxylamine-2-butyl-5-hydroxy-4[N-cyclohexanehydroxylamine-2-butyl-5-carboxyanhydride] and derivatives thereof.

### BACKGROUND ART

Matrix Metalloproteinases (MMPs) are a family of enzymes that degrade connective tissues in the body. These enzymes belong to the class of enzymes called proteases. Many cell types have this enzyme endogenously within them including fibroblasts, monocytes, macrophages and invasive tumor cells. In these tissues the enzyme functions to remodel tissue in the body. Changes occur in local tissues that stimulate the release of growth factors, cytokines and other signaling molecules. When these molecules stimulate the production of MMP, the cell can degrade the extracellular matrix including collagen, proteoglycans (protein core), fibronectin and laminin (Rodriguez J A, et al. Rev Esp Cardiol. 2007 September; 60 (9):959-67.). The joints in the body, connective tissues, cartilage and basement membranes have these components in them. Various diseases have hyperactivity of MMP enzymes in their etiology. Rheumatoid arthritis, osteoarthritis, COPD, stroke, tumor invasion and multiple sclerosis have MMP over-activity in their etiology. Targeting the MMP enzyme is a good therapeutic target for these disease processes (Wallach J et al. Biochimie 87: 241, 2005).

MMP enzymes use zinc and calcium elements to break down their targets including collagen, gelatin, etc. There are currently over ten enzymes that have been classified with sequence homology of 40-50% between subtypes (Fingleton B. Front Biosci 11: 479-491, 2006).

Collagen is the major structural protein in humans and mammals. It is a component of bone, cartilage, tendon and skin. There are 3 types of collagen in the Human body, type 1, 2 and 3. When collagens are degraded by the MMP enzymes, the by-products are then degraded by other enzymes. This process is ubiquitous throughout the body and plays a role in angiogenesis. (Handsley M M, et al. Int J Cancer 115: 849-860, 2005).

Techniques have been described for the quantification of MMP activity. Assay kits are available commercially for activity determination. (Roomi M W et al, 2003)

MMP inhibitors will provide useful treatment for diseases associated with breakdown of extracellular matrix such as multiple sclerosis, diabetes, arthritic diseases (arthritis and osteoarthritis, periodontal disease, tumor invasion and metastasis, and aberrant angiogenesis. The type of tissue involved will influence the type of inhibitor that you will have to use. For example, in a disease process that involves the joints, such as a arthritis, will benefit from a MMP inhibitor inhibiting Collagenase-1 and Collagenase-3 (Green M J et al. Rheumatology 2003; 42:83-88.). Administration of an MMP inhibitor that blocks these enzymes would be effective in stopping the disease process and provide therapeutic benefit. Inhibitors of these enzymes would also benefit diseases of soft tissue remodeling (Stamenkovic I. J Pathol. 2003 July 200 (4):448-64).

Inhibitors of MMP are also known to substantially inhibit the release of TNF, tumor necrosis factor. TNF is involved in disease processes as acute infections, shock states, vascular restenosis, aneurismal disease, autoimmune diseases, etc. Administration of these agents will be beneficial in these diseases. These disease processes also affect other biological processes involved in inflammation such as IL-6, M-CSF, Las Ligand and ICAM-1 (Konttinen Y T et al. Ann Rheum Dis 1999; 58:691-697).

Those familiar with the art will recognize that the. MMP enzyme is a ubiquitous type of enzyme in the body. Its biological target, Collagen and connective tissue in the body, is constant in the body. Sites and affinity for the target may change between classes of enzyme, however, affinity for biological target of a given enzyme e.g. MMP will be constant throughout the body (barring synthesis difference in the tissues). Therefore, if a compound inhibits MMP 9 at one site in the body, it will inhibit it at another site with that same affinity (Ramnath N, et al. Curr Oncol Rep 6: 96-102,2004).

Different tissues will allow drugs to diffuse into them at different concentrations and differences in the concentration gradient may be established in the body by differences in the tissues themselves to the drug. Therefore, for a given concentration of drug administered to the patient, in the short term, concentrations of drug may be higher in some tissues than others. With repeated dosing this concentration gradient is expected to diminish. Therefore, to target a specific location for desired action is not possible given ubiquitous enzyme location and affinity.

Utilizing multiple enzymes to activate the drug, it becomes possible to differentiate tissues with different metabolic/functional characteristics and enzyme profiles. Hence an activated compound can be used to target specific tissues and is superior to its non-targeted counterpart where desired enzyme is ubiquitous throughout the body.

Tolerability refers to a patient's ability to take the drug without side effects impairing the drugs ability to be taken. When side effects of a drug are to severe, patients cannot tolerate a drug and the drug is thus ineffective. An ideal drug is tolerated by a patient well and the drug has its biological effect in its target tissue. Poorly tolerated drugs have significant side effects that result in the patient having poor health and cannot thus take the drug for its biological activity.

Tolerability is proportional to a compounds biological activity outside of the desired tissue or area of activity. When a compound interacts with normal or beneficial processes in the body the patient will be adversely affected by the compound.

Compounds have been used prior in clinical trials in humans have had significant side effects and were not tolerated by patients. Prior compounds had poor tolerability. Specifically, the compounds caused tendonitic side effects such as joint pain in patients that were dosed with the compounds. This tolerability likely resulted from the compounds inhibiting beneficial collagen synthesis and remodeling in human joint cavities at doses that were high enough for biological activity (Skiles J W, et al. Curr Med Chem 2004 November; 11 (22):2911-77, Beckett, et al., Drug Discovery Today, 1996, 16-26, vol 1, No 1., Whittaker M, et al. Chem Rev. 1999 Sep. 8; 99 (9):2735-76, Michaelides, M R et al. Curr Pharm Design 5, 787-819, 1999, Hoekstra R, et al. The Oncologist 2001; 6:415-427)

Carboxypeptidase enzyme uses nascent water molecules to serve as electron donors to carboxypeptide covalent bonds. See FIG. 1 below. (FIG. 1 depicts cleavage of the bond by the enzyme). This enzyme catalyzes the reaction of carboxyl groups on bioactive molecules.

FIG. 1: Carboxypeptidase reaction

### FIG. 1

Those familiar with the art will appreciate that a compound might be split by carboxypeptidase from an inactive compound into a biologically active molecule. Using this bioactivation for an anticancer drug would be beneficial as the drug would only be active in tissues with carboxypeptidase activity. Similarly, the enzyme activity could target the drugs activity in the body.

Bioactivation has been reported for compounds with anticancer activity. For example, carboxylesterases play a critical role in the bioactivation of the anticancer prodrug irinotecan {7-ethyl-10[4-(1-piperidino)-1-piperidino] carbonyloxy-camptothecin; CPT-11} into its active metabolite SN-38 (ethyl-10-hydroxycamptothecin).

Mammalian enzymes have been described which cleave substrates which are tissue specific. Human gut endothelium is rich in carboxypeptidase enzyme. Carboxylesterase activity is found in serum, liver, intestine and other sites (Kaneda N et al. Cancer Res 1990; 50: 1715-1720). Different isoforms of enzyme are present with variable enzymatic velocity in cleaving irinotecan. Genetic variability of carboxylesterase expression and/or activity is suspected, as variations of SN-38 levels are observed from individual to individual for a given dose of irinotecan (Ohe Y et al. J Natl Cancer Inst 1992; 84: 972-974).

Many chemical compositions with intended MMP activity in the prior art. Numerous targets have been disclosed, such as MMP-2 and TNF binding with a dihydroxy-hydroxyethyl-tetrahydrofuran-pyrimidine-dione in U.S. Pat. Nos. 6,930,097 and 6,683,060 assigned to Advanced Gene Technology Corp. Also described for broad MMP inhibition are pyrimidine-trione inhibitors as described in U.S. Pat. No. 6,579,982 assigned to Pfizer inc. MMP inhibitors with claimed reduced side effects is described in U.S. Pat. No. 6,818,622 and heterocylic compounds with MMP inhibitory activity in U.S. Pat. No. 6,063,786 are both described and assigned to Darwin discovery ltd.

U.S. Pat. No. 6,541,489 and U.S. Pat. No. 6,380,258 assigned to G. D. Searle & Company for Sulfonyl divalent aromatic or heteroaromatic ring, hydroxamic acid compounds and aromatic sulfone hydroxamic acid inhibitors, citing inhibitory activity against MMP-2, MMP-9, and MMP-13, with less inhibition of MMP-1.

U.S. Pat. No. 6,352,976 assigned to Aventis Pharmaceuticals Inc. describes mercaptoacetylamido dipeptide carboxylic acids as MMP inhibitor. U.S. Pat. No. 5,872,152 assigned to British Biotech describes hydroxamic acid MMP inhibitors, for use in angiogenesis with antitumor activity. U.S. Pat. No. 5,847,153 assigned to Pharmacia & Upjohn Company describes beta-sulfoynl hydroxamic acids useful as MMP inhibitors, with stromelysin, collagenase, and gelatinase inhibition. Other hydroxamic acid based inhibitors are described in U.S. Pat. No. 4,599,361 and U.S. Pat. No. 4,743,587 assigned to G. D. Searle & Co., U.S. Pat. No. 4,681,894 assigned to Ortho Pharmaceutical Corporation and U.S. Pat. Nos. 6,118,001 and 6,465,468 assigned to Darwin discovery ltd. Amino acid derivatives were described in U.S. Pat. No. 4,558,034 assigned to The Board of Trustees of the University of Kentucky for bacterial collagenase inhibition and in U.S. Pat. No. 4,918,105 assigned to SA Laboratoire Roger Bellon. Peptide derived compounds for collagenase inhibition were described in U.S. Pat. No. 5,616,605 assigned to Research Corporation Tech., Inc. N-acyl-L-amino acid carboxamide and its synthesis are described in U.S. Pat. No. 5,892,112 assigned to Glycomed Incorporated and the University of Florida for MMP inhibition. Sulfonyl aromatic hydroxamic acid compounds inhibiting matrix metalloproteinase activity and/or aggrecanase activity were described in U.S. Pat. No. 6,696,449 assigned to Pharmacia Corporation.

Other compounds for Matrix Metalloproteinase inhibition are described in U.S. Pat. No. 5,932,595 assigned to Syntex (U.S.A.) Inc. and Agouron Pharmaceuticals, Inc. MMP 13 inhibitors have been described utilizing thiol sulfone derivatives metalloproteinase as in U.S. Pat. No. 6,013,649 assigned to Monsanto Company. Other compounds with MMP inhibitory activity are described in U.S. Pat. No. 5,183,900 U.S. Pat. No. 5,189,178 by Galardy et al. Thiol based MMP inhibitors are described in U.S. Pat. No. 4,595,700 assigned to G. D. Searle & Co.

Inhibition of undesired MMP activity is desired for a number of disease processes. Activation of a MMP inhibitor compound at the site of aberrant MMP activity would be beneficial to the patient by limiting the adverse effects encountered during treatment.

### DISCLOSURE OF THE INVENTION

This disclosure provides new compounds which are useful as matrix metalloproteinase inhibitors. Furthermore, the compounds described herein are beneficial in conditions which characterized by undesirable activity of these enzymes

Accordingly, in one aspect the invention is disclosed as compounds such preferably shown in FIG. 2. One example of said invention may be described as a 1-R-1-hydroxyl-2R-Propanoate-5[1S,2S¹]-propanoic acid core. Additional chemical entities can be synthesized incorporating the chemical core structure shown in FIG. 3.

FIG. 2:

### FIG. 2

FIG. 3:

### FIG. 3

Alternatively, those familiar with the art will recognize that R, R1, S and S1 may be part of a cyclic ring structure such as depicted in FIG. 4: Whereby R or R1, S or S1 is a Carbon, Nitrogen or Sulfur atom Z or Z1 and B or B1 is independently H or an alkyl, amino acid residue or amide thereof. Alternatively, Z or Z1 and B or B1 may constitute an fused, unsubstituted, cyclic ring structure, a cyclic amine or heterocyclic amine, or heavy metal, halogen or oxygen species.

FIG. 4:

### FIG. 4

An example presented of an preferred molecule for MMP inhibition is N-cyclohexanehydroxylamine-2-butyl-5-hydroxy-4[N-cyclohexanehydroxylamine-2-butyl-5-carboxyanhydride] as depicted in FIG. 5.

FIG. 5:

### FIG. 5

The purpose of said chemical structure is to provide a scaffold for a cellular enzyme to split the carboxypeptide bond in the structure depicted in FIG. 3. This process utilizes carboxypeptidase cellular enzyme selectively cleaving the carbon backbone at the enol oxygen, end products are depicted in FIG. 6. This cleavage produces two molecules from the parent. As such the molecule reveals two daughter molecules of identical or similar structure. For purposes of molecular therapeutics, this in essence doubles the local dose of the therapeutic molecule. Of note, the parent molecule in and of itself has minimal therapeutic activity. The daughter-split products have MMP inhibitory activity that is desired in target tissues or cells.

FIG. 6:

### FIG. 6

Those familiar with the art will appreciate the daughter molecules may be of separate chemical composition or of identical compounds. The invention, specifically R, R1, S and S1 may be be synthesized into a conjugate structure linked by the carboxypeptide bond in FIG. 1. Non-identical compounds can be used in compound synthesis to synergistically act for pharmacologic benefit. Identical daughter compound end product facilitate a doubling of the dose in tissues that are targeted with the enzyme, allowing a doubling of the dose locally with minimal effect outside the targeted region.

The invention can also be applied whereby R, R1, S or S1 is a compound in complementary chemical structure to FIG. 3. As such, any such derivative will possess a bond with structure depicted in FIG. 3. Therefore, enzymatic breakdown of the side chain, in a manner consistent with enzyme breakdown of FIG. 3, yields equal compound derived from the parent. Respectively, different compounds may be derived from the splitting of R, R1, S, S1 by enzymatic action. An example of such a compound is depicted FIG. 7 below.

FIG. 7:

### FIG. 7

As such, dose of active compound derived from parent compound can be increased by (number of formula 1 centers+1) in areas of the body where enzymatic action is present. As such, tissues with enzymatic activity will have active compound concentrations increasing in concentration with each replication of daughter compound in the invention.

The key characteristic compounds of FIG. 3 that determine their intrinsic value is their ability to be biologically inert when not at their site of action, and biologically active at their site of desired activity. This function is accomplished by enzymatically cleaving the molecule at the site of action by enzymes preferentially expressed in tissues at the site of action. With invention compounds as shown in FIG. 3, the native enzyme principally involved in cleaving the molecule is carboxypeptidase.

Reference is made to the molecule of invention shown in FIG. 8, carboxypeptidase enzyme cleaves the molecule at site X into 2 distinct molecules. With modification of constituent structures labeled R and S respectively, the molecule can be cleaved into 2 separate, distinct chemical entities or it can be cleaved into 2 identical moieties. Those familiar in the art will respect that the molecules desired from cleavage can have biological benefit if the end products are not identical in structure.

FIG. 8:

### FIG. 8

### Liposomal Preparations

It is possible to prepare and administer medicinal compounds in a liposomal preparation. Liposomal preparations consist of a medicinal compound preparation, which is then applied with an outer shell encasement consisting of a lipid compound or compounds.

Such preparations provide encasement of a bioactive molecule within an inert shell consisting of lipid and/or other composition. The construct functions by slowly releasing bioactive compound from its core, or by mating with cellular membranes, fusing and releasing active compound into the core of the cell. Either means provides a steady release of bioactive compound into the body.

The invention may be encased in a liposome for administration. This will facilitate the slow release of the invention for biological activity. Those familiar with the art will recognize the invention can be applied to a multitude of pharmaceutical delivery modalities such as but not limited to conjugation to an antibody, conjugation to another molecule for localization, etc.

The compounds of invention as shown in FIG. 3 are advantageous to prior MMP inhibitor compounds in their higher tolerability. It is a significant advantage of compounds of formula 1 to be selectively activated in target tumor and tissues by endogenous enzyme found in tumor or target tissue. Selective activation at the site of the undesired MMP activity, such as in tumor and not in other tissues such as joint capsular tissue where there is little carboxypeptidase activity, provides improved tolerability with pharmacologic treatment. Having greater tolerability gives advantage to one compound over the other in application to human health.

### Therapeutic Use of Compounds of the Invention

As presented in background art, a number of diseases are known to result from unregulated MMP activity and undesired MMP activity. These include tumor metastasis, rheumatoid arthritis, aneurism formation, skin inflammation, and the like. Hence, compounds that impair MMP activity and function are beneficial when used as therapy for these conditions by lowering MMP activity.

The invention is useful in the prophylaxis and treatment of disorders characterized by undesirable MMP activity. Specifically, invention this relates to tumor invasion and growth. Application of the invention to tumor However, those familiar in the art will recognize that application of the invention may also be of benefit in other biological processes that incorporate undesired MMP activity, such as inflammatory disorders, infectious processes, vascular disorders, reproductive disorders, etc.

Administration of the compounds of Formula I or their salts, in their pure form or in appropriate pharmaceutical composition, can be carried out via any of the acceptable modes of administration or agents for serving similar utilities.

Thus, administration can be orally, nasally, parenterally, topically, transdermally, rectally, in the form of solid, semi-solid, lyophilized powder, or liquid dosage forms. These are examples and are not mutually inclusive in the art. Dosage forms are similarly described in example such as tablet, suppositories, pills, soft elastic and hard gelatin capsules, powders, solutions, suspensions, aerosols, or the like.

Administration is preferred for simple administration of precise dosages. The compositions will include a conventional pharmaceutical carrier, and a compound or formula I as the active agent, and in addition may include other medicinal agents, pharmaceutical agents, carriers, adjuvants, etc.

Generally, depending on the intended mode of administration, the pharmaceutically acceptable compositions will contain about 1% to about 99% by weight of a compound(s) of Formula I, or a pharmaceutically acceptable salt thereof, and 99% to 1% by weight of a suitable pharmaceutical excipient. Preferably, the composition will be about 5% to 75% by weight of a compound(s) of Formula I, or a pharmaceutically acceptable salt thereof, with the rest being suitable pharmaceutical excipients.

The preferred route of administration is oral, using a convenient daily dosage regimen which can be adjusted according to the degree of severity of the disease-state to be treated. For such oral administration, a pharmaceutically acceptable composition containing a compound(s) of Formula I, or a pharmaceutically acceptable salt thereof, is formed by the incorporation of any of the normally employed excipients, such as for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such compositions take the form of solutions, suspensions, tablets, pills, capsules, powders, sustained release formulations, and the like.

Alternatively, intravenous and parenteral administration may be preferable in hospital settings for precise administration of the invention to patients. The invention may be formulated in aqueous solution as a chemical entity with stabilizing salt suspension, carbohydrate, or other formulation. Liposomal preparations may be administered in intravenous or parenteral formulation. Those familiar in the art will respect variation in formulation of the art.

Preferably such compositions will take the form of capsule, caplet or tablet and therefore will also contain a diluent such as lactose, sucrose, dicalcium phosphate, and the like; a disintegrant, such as croscarmellose sodium or derivatives thereof; a lubricant such as magnesium stearate and the like; and a binder such as a starch, gum acacia, polyvinylpyrrolidone, gelatin, cellulose ether derivatives, and the like.

The compounds of Formula I, or their pharmaceutically acceptable salts, may also be formulated into a suppository using, for example, about 0.5% to about 50% active ingredient disposed in a carrier that slowly dissolves within the body, e.g., polyoxyethylene glycols and polyethylene glycols (PEG); e.g., PEG 1000 (96%) and PEG 4000 (4%).

Liquid pharmaceutically administrable compositions can, for example, be prepared by dissolving, dispersing, etc., a compound(s) of Formula I (about 0.5% to about 20%), or a pharmaceutically acceptable salt thereof, and optional pharmaceutical adjuvants in a carrier, such as, for example, water, saline, aqueous dextrose, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Actual methods of preparing such dosage forms are known, or will be apparent, to those skilled in this art; for example, see Remington's Pharmaceutical Sciences, 18th Edition, Mack Publishing Company, Easton, Pa. (1990). The composition to be administered will, in any event, contain a therapeutically effective amount of a compound of Formula I or a pharmaceutically acceptable salt thereof, for treatment of a disease-state alleviated by the inhibition of matrix metalloproteinase activity in accordance with the teachings of this invention.

The compounds of Formula I or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-state, and the host undergoing therapy. Generally, a therapeutically effective daily dose is from about 0.014 mg to about 14.3 mg/kg of body weight per day of a compound of Formula I or a pharmaceutically acceptable salt thereof; preferably, from about 0.07 mg to about 5 mg/kg of body weight per day; and most preferably, from about 0.14 mg to about 1.4 mg/kg of body weight per day. For example, for administration to a 70 kg person, the dosage range would be from about 1 mg to about 1.0 gram per day of a compound of Formula I or a pharmaceutically acceptable salt thereof, preferably from about 5 mg to about 300 mg per day, and most preferably from about 10 mg to about 100 mg per day.

The inhibitors of the invention are targeted to the site of desired inhibition of MMP activity by possession of carboxypeptidase enzyme activity. Tissues and tumors with carboxypeptidase enzyme are able to cleave the parent compound, an example of invention in formula 2, into daughter compounds with MMP inhibitory activity.

Invention is applied dissolved in aqueous solution or in liposome based delivery intravenously. Compound is dispersed in bodily fluids to which it maintains a steady state. The dose may be applied in a single constant application or may be applied in a rapid administration. At the tissue level invention is available to tissues without active carboxypeptidase enzyme and may be weakly inhibitory to MMP enzyme, pending variation in active site MMP enzyme between different tissues. At tumor sites, tissue levels of MMP are upregulated for tumor invasion. When tumor tissue also possesses carboxypeptidase enzyme, the compound is split into active daughter compound and MP inhibition is facilitated locally. Tumor tissue may also be expressing MMP enzyme for tumor related vascular remodeling. Increased local concentration of invention split products will inhibit this enzyme activity as well, and impair tumor related vascular remodeling. At distant sites, local tissue concentration is lower and normal vascular biology is unaffected.

MMP activity is higher tumor tissues during invasion of adjacent tissue structures. Enzymes facilitate the degradation of basement membrane, collagenases in particular are advantageous to tumor tissue for this process. Administration of invention derivatives during this process blocks MMP activity and consequently prevents tumor invasion and metastasis. Contingent upon this is carboxypeptidase enzyme expression in tumor tissue itself being particularly abundant in gastrointestinal tissue and tumors of the like. Tumors can remodel local vasculature to supply greater blood flow to tumor tissue. Tumor MMP related remodeling of vasculature is inhibited by the invention and its derivatives, therefore limiting nutrient delivery to the tumor. Administration of the invention to such tumors is thus therapeutically beneficial.

Vascular tissue is remodeled by MMP enzyme degrading the connective tissue and basement membrane lining stroma of the vessel. Aneurisms and other adverse remodeling of vascular tissue are the result of undesired MMP activity. Specifically, MMP degradation of the vascular tissue leads to thinning of the wall and aneurism formation. Inhibition of this undesired activity will act as prophylaxis for undesired vascular remodeling, specifically affording treatment and prophylaxis for aneurism formation.

Utilization of a liposome delivery formulation may be applied to the invention. Application will prolong the half life of invention in the body. This allows areas of higher metabolic demand and blood flow, to be exposed to the compound in greater frequency compared to adjacent tissues. Respectfully, invention is supplied to tumor tissue and inflammatory tissue with greater abundance than comparable tissue, with greater effect on said tissues. Therefore the invention can be partially targeted to desired site of action with liposome delivery application.

Clinical use of the invention is also advantageous in infectious disease, inflammation, ulcerations, rheumatoid arthritis, reproductive functions and the like.

### The Compound Is Synthesized In the Following Way

Finalized invention for administration is synthesized in a 3-step process. The first step, step 1, consists of synthesis of the active parent compound. The second, step 2, consists of synthesis of liposome delivery compound if one is to be used with the invention. The third step entails mixing the two entities into a finalized liquid suspension, step 3 if a liquid suspension is to be used for administration. Those familiar with the art will appreciate a multitude of ways to synthesize invention. An example of synthesis therefore follows:

### Example Step 1

### Synthesis of Invention Parent Compound

Reaction scheme shown in FIG. 9 below. 2,6-hydroxy-3,5-chloro-hexane and 2 methyl-3 chloropropane are mixed with acetic acid, disodium carbonate in aqueous suspension at 200 degrees centigrade for 2 hours with gentle agitation (Step 2). 1-carboxy-2,-hydroxyl-5-butyl-cyclohexene product is then separated with washing C8-C12 alkane solution at room temperature for 30 minutes, hydrocarbon layer is removed with dissolved compound. 2,3,6,hydroxy-5-isopropylhexane reaction by product is also removed in the hydrocarbon layer after washing. Aqueous layer is discarded. This product consists of 2,3,6-hydroxy-5-isopropylhexane and 1-carboxy, 2,4-hydroxyl,5-butyl-cyclohexane dissolved in hydrocarbon layer as solution B. Solution B is reacted with aqueous carboxylic acid solution (1 molar) and hydroxynitrocarboxic acid (2 molar) at 200 degrees centigrade for 20-30 minutes in 0.2 Molar Sodium hypochlorite solution (Step 5 and 6) with agitation. Resultant reaction is a solution of N-cyclohexanehydroxyamine, 2-butyl, 5-hydroxyl, 4-carboxylic acid and N-hydroxycarbohydroxamic acid dissolved in hydrocarbon layer. Solution is then cooled and 0.5M methyl alcohol is added to the solution. Solution is agitated for 30-60 minutes after which the hydrocarbon layer is removed. Aqueous solution is chilled to 0 degrees centigrade with solid product collected on the bottom of the flask. Solution is removed from the flask whereby 80 percent of the solution is removed and discarded. Resultant supernatant is mixed with hydrochloric acid solution (0.5 molar) in aqueous solution of 5% ethyl alcohol and heated at 150 to 180 degrees for 1 hour with gentle agitation (step 7). Solution is cooled to room temperature (60-70 degrees) and pH stabilized to 6.5 to 7.2. Solution is cooled to 0 degrees centigrade and gently warmed to 10 degrees centigrade. Powder precipitate is collected as aqueous solution is decanted and powder removed with a fine stainless steel grate for drying on a flat metal plate at a temperature of 25 degrees centigrade under a fume hood and charcoal filter. Crystal precipitate is re-suspended in a buffered solution of 0.9% sodium chloride and preservative before solution is vacuum desiccated and prepared for administration.

FIG. 9: Reaction scheme 18

### FIG. 9

### Step 2: Synthesis of Liposomal Delivery Vector For Delivery of Invention

This synthesis applies if the invention is to be administered via liposomal delivery. Overall, the preparation of liposomes entails preparation of large vesicles which envelope the compound of desired delivery. Large vesicles are then sonicated into smaller vesicles of desired size. The Morrisey method is described herein however other methods can be applied. (Morrissey Lab Protocol for Preparing Phospholipid Vesicles (SUV) by Sonication. Protocol from James H. Morrissey, Dept. of Biochemistry, University of Illinois at Urbana-Champaign, Urbana, Ill. 61801, USA)

In a controlled environment, PL mixture L-alpha-Phosphatidylcholine, egg10 or 25 mg/ml, L-alpha-Phosphatidylserine, bovine brain-Na salt 10 mg/ml, L-alpha-Phosphatidylethanolamine, bovine Liver 10 mg/ml, is dried utilizing nitrogen or argon gas. The combination of phospholipids can be varied and is modified easily to those familiar in the art. 1:1 combinations consisting of Phosphatidyl serine and Phosphatidyl Choline are frequently employed. Once dried to 5% humidity, the remaining moisture is removed with vacuumed desiccation for an additional 60 minutes under high vacuum. (This is to remove any residual chloroform from manufacturing.) To the dried-down PL, add 2.6 ml/mole room temperature HBS solution (100 mM NaCl, 20 mM Hepes/NaOH buffer, pH 7.5, 0.02% (w/v) sodium azide) and cover top of container. Let sit 1 hr at room temperature. The tube is then Vortexed vigorously to completely resuspend the PL. A milky, uniform suspension will be resulting.

A bath sonicator is filled with room temperature water (Bath sonicator, example: Model G112SPIG from Laboratory Supplies Co., Inc.). The tube containing the PL suspension is placed in the bath. The liquid level inside the PL container should equal that outside the tube. The container is Sonicated until the suspension changes from milky to nearly clear (i.e., only very slightly hazy) in appearance. Check every 10 min; it will usually take between 10 and 30 min total sonication time. The bath is not allowed to overheat and is not drained until it has completely cooled. The final product is stored at 4 degrees C. The result is a suspension of small unilamellar vesicles (SUV) containing a total of 1 mM phospholipid in HBS.

Confirmation of the phospholipid concentration by assaying total phosphorus content is possible. The method can be easily scaled. Simply make a larger batch, and sonicate in a larger container (the sonication time may be longer). To make very large amounts, sonicate in small batches of product. Respectively, variations of this protocol can be made by those familiar with the art. Variation in the composition and ratio of Phospholipid may be employed in the formulation process to vary composition properties according to desired biologic activity.

### Step 3: Combination of the Two Preparations Into A Liposomal Formulation

This step in synthesis applies if the invention is to be administered via liposome. Active compound is prepared in a buffered solution of sodium, chloride, potassium, calcium with respective preservative, an example being sodium citrate maintained at 40 degrees centigrade. Compound is prepared according to the specification in Step 1 Above. This is added to Phospholipid (PL) in a sterile aliquot in lieu of HBS solution in Step 2 above. Resultant mixture is allowed to sit for approximately 1 hour under nitrogen or argon gas. Active compound and PL are then vortexed vigorously under argon or nitrogen gas for approximately 10 minutes to completely suspend the compound and PL. This aliquot is then placed in the sonicator as described in Step 2 above. This aliquot is then sonicated for approximately 10 to 30 minutes to provide unilamellar vesicles containing the Active compound. Sodium and electrolyte composition of the final solution are checked and adjusted as needed for a final concentration of 0.9% sodium chloride prior to administration. This compound is stored at 4 deg. C. until needed and is administered intravenously for therapeutic effect.

### BRIEF DESCRIPTION OF THE DRAWINGS

FIG. 1 shows the reaction scheme of Carboxypeptidase enzyme, specifically how the enzyme utilizes water molecules to serve as electron donors during cleavage of carboxypeptide bonds.

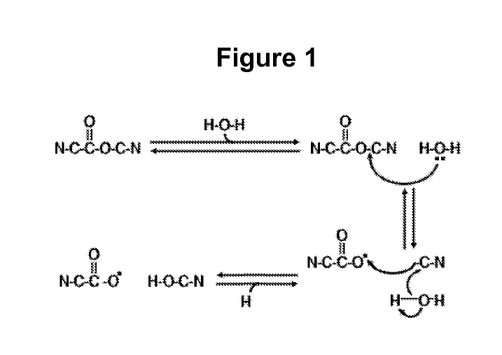

FIG. 2 shows one example of the invention, N-cyclohexanehydroxylamine-2-butyl-5-hydroxy-4[N-cyclohexanehydroxylamine-2-butyl-5-carboxyanhydride containing the chemical core structure of the invention.

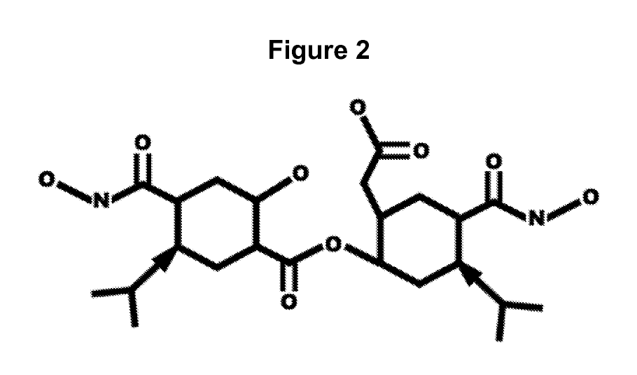

FIG. 3 shows the chemical core structure of the invention.

FIG. 4 shows the chemical core structure of the invention, whereby R, R1, S and S1 are a part of a ring structure.

FIG. 5 shows N-cyclohexanehydroxylamine-2-butyl-5-hydroxy-4[N-cyclohexanehydroxylamine-2-butyl-5-carboxyanhydride], a preferred embodiment of the invention.

FIG. 6 shows two daughter molecules of chemical core of invention after selective cleavage of invention carbon backbone at the enol oxygen by carboxypeptidase cellular enzyme.

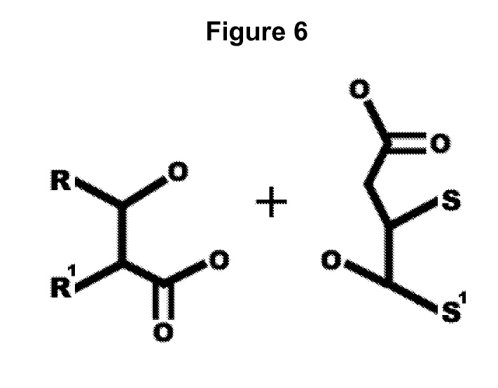

FIG. 7 shows a chemical compound preferred embodiment of invention with two carboxypeptide bonds in the preferred embodiment molecule.

FIG. 8 shows the site on the invention molecule where carboxypeptidase enzyme binds to the invention molecule and cleaves it.

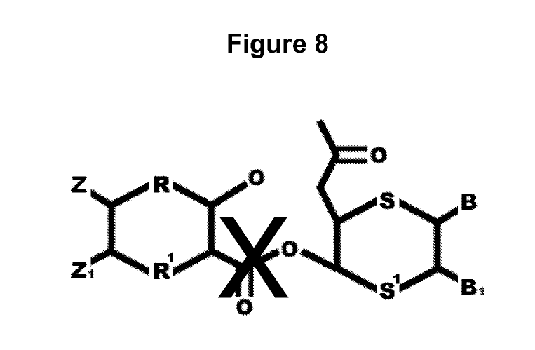

FIG. 9 shows a chemical reaction scheme for synthesis of invention compounds.

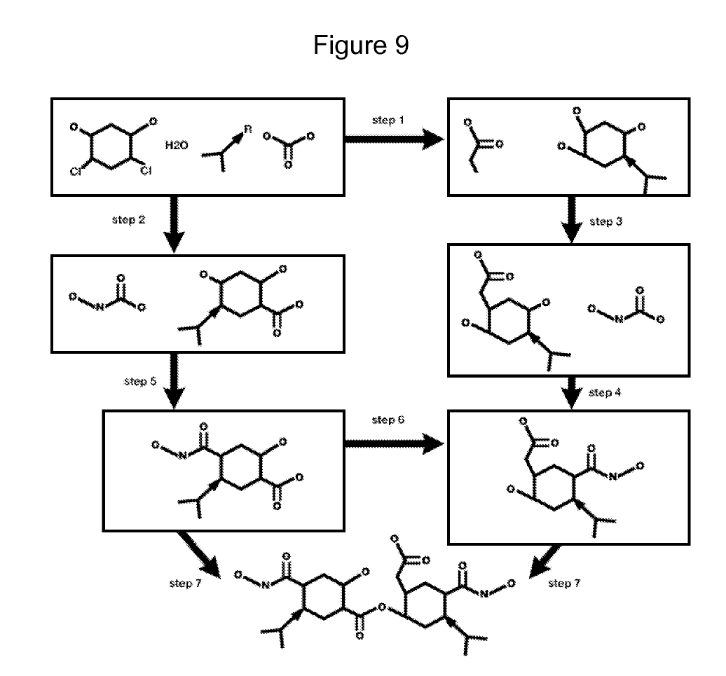

I claim the following:

1. A method of inhibiting cancer cell proliferation and/or promoting tumor regression in a patient comprising administering an effective amount of an exogenous matrix metalloprotease (MMP) inhibitor to said patient sufficient to inhibit cancer cell proliferation and/or tumor regression.

2. The method of claim 1, wherein tumor is a solid tumor that is of breast, ovarian, neuroectodermal, lung, colorectal, gastric, renal, pancreatic, cervical or prostate carcinoma, a melanoma, a sarcoma, or an ascites thereof.

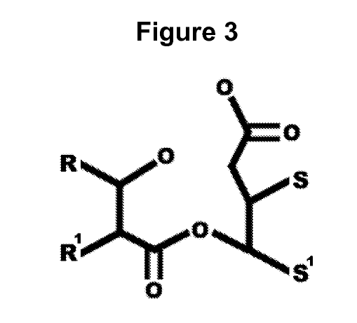

3. The method on claims 1 wherein MMP inhibitor contains compounds with the formula of C7O5R, R1, S, S1 as depicted in FIG. 3

4. Compounds that derive their matrix metalloproteinase inhibitory activity in cellular tissue or tumor tissue by enzymatic cleavage of the C—O bond as depicted in FIG. 3.

5. Compounds and derivatives of claim 3 with activity involving inhibition of matrix metalloprotease activity in cellular tissue in the body

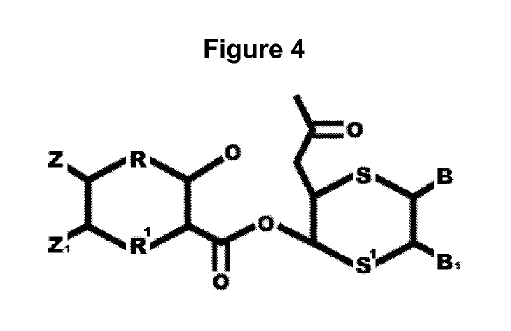

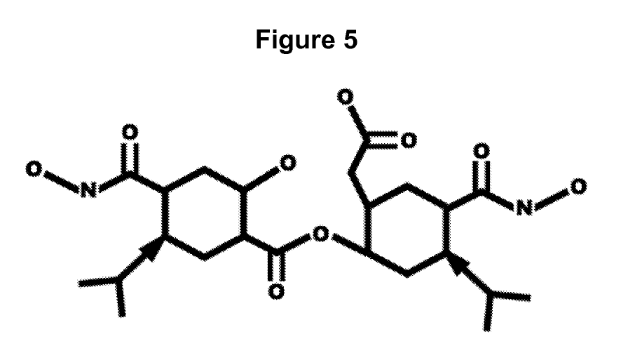

6. Derivatives of invention compounds described in claim 3 that constitute additional ring structure as preferred in FIG. 4 and FIG. 5.

7. Ligation of compounds to R, R1, S, and S1, as claim 3 above, adjuncts on above compounds that biological activity other than inhibition of matrix metalloprotease inhibition, which function in synergistic means in the body for therapeutic means.

8. Compounds of claim 3 above whereby R, R1, S and S1 are part of a ring structure, being atoms consisting of C, S, N, or O.

9. Administration of invention compounds of claim 3 by formulation that facilitates oral, intravenous, transdermal, transmucosal or other means.

10. Invention compound derivatives of claim 3 that are delivered via conjugated antibody, bound substrate, bound in chemical salt, or other.

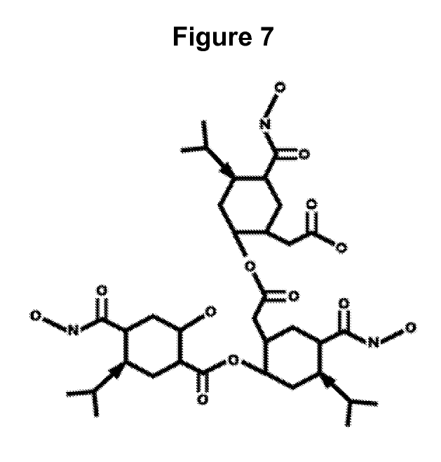

11. Preferred compounds and derivatives of claim 3 as described in FIGS. 4, 5 and 7 for use in inhibiting matrix metalloprotease activity.

12. Invention compounds as described that are administered as inactive compound and activated specifically within tissues of undesired matrix metalloprotease activity for desired effect.

13. Invention compounds and their derivatives by which their biologic activity takes effect within tumor tissue as claim 12 and results in increases in concentration of beneficial drug in tissue concentration above serum levels.

14. Invention compounds and their derivatives as described in claims 12 in which tumor tissue enzymes facilitate the biologically beneficial properties of said invention.

15. Chemical Synthesis of invention in claims 12 as described.

16. Compounds described above in claims 12 that are composed in a micelle or liposomal preparation, for application to the body.
